(19) United States
(12) Patent Application Publication
(10) Pub. No.: US 2013/0310679 A1
(43) Pub. Date: Nov. 21, 2013

(54) 3D TRANSURETHRAL ULTRASOUND SYSTEM FOR MULTI-MODAL FUSION

(71) Applicants: Shyam Natarajan, Cupertino, CA (US); Martin Culjat, Los Angeles, CA (US); Rahul Singh, Palo Alto, CA (US)

(72) Inventors: Shyam Natarajan, Cupertino, CA (US); Martin Culjat, Los Angeles, CA (US); Rahul Singh, Palo Alto, CA (US)

(73) Assignee: The Regents of the University of California, Oakland, CA (US)

(21) Appl. No.: 13/868,863

(22) Filed: Apr. 23, 2013

Related U.S. Application Data
(60) Provisional application No. 61/637,166, filed on Apr. 23, 2012.

(51) Int. Cl. A61B 8/00; A61B 6/03; A61B 5/055; A61B 8/12; A61B 8/08

(52) U.S. Cl. CPC A61B 8/445; A61B 8/12; A61B 8/466; A61B 8/5261; A61B 8/5246; A61B 5/055; A61B 6/032; A61B 6/037; A61B 8/4488; A61B 8/4494
USPC 600/411; 600/463; 600/440; 600/427

(57) ABSTRACT

A three-dimensional transurethral ultrasound system includes a transurethral ultrasound probe, an ultrasound data processor configured to communicate with the transurethral ultrasound probe to receive ultrasound signals from the transurethral ultrasound probe and to output ultrasound imaging signals for three-dimensional ultrasound images of at least a portion of a patient's prostate, and a display system configured to communicate with the ultrasound data processor to receive the ultrasound imaging signals and to render three-dimensional ultrasound images of the at least the portion of the patient's prostate from the ultrasound imaging signals.

### CROSS-REFERENCE OF RELATED APPLICATION

This application claims priority to U.S. Provisional Application No. 61/637,166 filed Apr. 23, 2012, the entire contents of which are hereby incorporated by reference.

This invention was made with U.S. Government support of Grant No. W81XWH-10-1-0156, awarded by the U.S. Department of Defense. The U.S. Government has certain rights in this invention.

### BACKGROUND

1. Field of Invention

The field of the currently claimed embodiments of this invention relates to ultrasound systems and components, and more particularly to three-dimensional transurethral ultrasound systems and components.

2. Discussion of Related Art

Prostate cancer is the most common non-skin cancer found in men in the U.S. and is the second leading cause of cancer death in men, slightly behind lung cancer [1]. Each year approximately 180,000 men are diagnosed with prostate cancer and approximately 31,000 succumb to the disease [2]. However, a 4% per year decrease in deaths caused by prostate cancer in the U.S. was reported from 1994-2004, and was attributed to early detection and advancements in treatment [3]. Improved imaging techniques may not only further reduce deaths by increasing the ability to detect prostate cancer, but may also improve surgical outcomes and monitoring.

Various imaging techniques have been applied to the early detection of prostate cancer, to pre-operative treatment planning, intra-operative image guidance, and post-operative quality assurance, including magnetic resonance (spectroscopy) imaging (MRI/MRSI), computed tomography (CT), X-ray fluoroscopy, and transrectal ultrasonography (TRUS). MRI has been used to provide detailed information of prostatic, periprostatic, and pelvic anatomy, and MRSI has the ability to differentiate between normal and distorted tissue metabolism [4]. CT is commonly used to determine prostate boundary and brachytherapy seed location post-operatively [5]; however, CT has limited accuracy and reproducibility in segmentation of the prostate boundary [6]. Both MRI and CT are limited by cost and are not practical as intra-operative imaging tools. Fluoroscopy is effective for needle guidance and radioactive seed placement, but not for imaging of the prostate [5].

Ultrasound has the advantage that it is low cost, non-ionizing, and has the ability to provide real-time imagery, and thus can be used for pre-, intra-, and post-operative imaging. TRUS is used for intra-operative needle guidance during radiation therapy, and has been shown in one study to detect a greater number of small tumors than a rectal-digital (finger) examination [7]. However, TRUS is limited in that it requires ultrasound energy to penetrate across the full volume of the prostate due to the external position of the transducer in the rectum (FIG. 1). Due to the increased penetration depth required, lower frequency transducers (5.0-7.5 MHz) are necessary to overcome acoustic losses, resulting in lower resolution imagery. The external vantage point of the TRUS transducer also results in shadowing, which may decrease image quality [5]. Some risks have been associated with TRUS guided needle biopsy, with one study reporting complications in 63.6% of patients, most commonly persistent hematuria [8].

Transurethral ultrasound (TUUS) has been proposed as an alternative to TRUS. By imaging the prostate from within (FIG. 2), this approach may enable higher frequency and therefore higher resolution imaging due to shorter required penetration depth, and at the same time eliminate shadowing effects that are prevalent in TRUS. TUUS was introduced nearly 20 years ago, and was fundamentally proven to be an effective tool in imaging of the urethral lumen, the urethral wall, the corpus spongiosum, and the corpora cavernosa [9]. However, there has been limited study of TUUS imaging of the prostate.

Between 2000 and 2004, a series of papers by D. R. Holmes et al. were published that demonstrated the potential capabilities of TUUS. These studies used commercially available intravascular ultrasound (IVUS) and intracardiac ultrasound (ICUS) catheter-based transducers, including rotational single-element transducers and linear phased array transducers [10, 11]. The authors performed in-vivo animal and human studies, and reported that the technique could be used to accurately localize radioactive seeds and prostate boundaries [5]. This technique also provided better spatial resolution and soft tissue differentiation than CT, as well as superior resolution and seed localization compared to TRUS [5]. However, the transducers were limited in their ability to provide 3D imagery, primarily due to the constant pullback velocity required in the rotational scheme [5] and the manual rotation required in the linear phased array scheme [10, 11]. The same group has also indicated in recent abstracts that a new linear array transducer was undergoing development and preliminary testing [12, 13]. All of the results have demonstrated the potential benefits that TUUS may provide for prostate imaging and monitoring; however no commercial TUUS transducers have been introduced to date. Therefore, there remains a need for improved three-dimensional transurethral ultrasound systems and components.

### REFERENCES

[1] American Cancer Society. Cancer Facts and Figures 2007. Atlanta: American Caner Society; 2007
[2] Denmeade S R and Isaacs J T, “A History of Prostate Cancer Treatment,” Nature Reviews Cancer, vol. 2(5), pp. 389-396, 2002.
[3] Ries L A G, Ries L A G, Melbert D, Krapcho M, Mariotto A, Miller B A, Feuer E J, Clegg L, Homer M J, Howlader N, Eisner M P, Reichman M, and Edwards B K (eds). “SEER Cancer Statistics Review, 1975-2004”, National Cancer Institute, Bethesda, Md., 2007.
[4] Coakley F V, Qayyum A, and Kurhanewicz J, “Magnetic Resonance Imaging and Spectroscopic Imaging of Prostate Cancer,” Journal of Urology, vol. 170, pp. S69-S76, 2003.
[5] Holmes D R, Davis B J, Bruce C J, and Robb R A, “3D visualization, analysis, and treatment of the prostate using trans-urethral ultrasound,” Computerized Medical Imaging and Graphics, vo. 27(5), p. 339 D, 2002.
[6] Dubois D F, Prestidge B R, Hotchkins L A, Prete J J, and Bice W S, “Intraobserver and interobserver variability of MR imaging- and CT-derived prostate volumes after trasnperineal interstitial permanent prostate brachytherapy,” Radiology, vol. 207(3), pp. 785-789, 1998.
[7] Carlsson P, Pedersen K V, and Varenhorst E, “Costs and benefits of early detection of prostatic cancer,” Health Policy, vol. 16, pp. 241-253, 1990.
[8] Rodriguez L V and Terris M K, “Risks and Complications of Transrectal Ultrasound Guided Prostate Needle Biopsy: A Prospective Study and Review of the Literature,” Journal of Urology, vol. 160, pp. 2115-2120, 1998.
[9] Pavlica P, Menchi I, and Barozzi L, “New imaging of the anterior male urethra,” Abdominal Imaging, vol. 26, pp. 180-186, 2003.
[10] Holmes D R and Robb R, “Trans-urethral ultrasound (TUUS) imaging for visualization and analysis of the prostate and associated tissues,” Proceedings SPIE Medical Imaging 2000: Visualization, Image-Guided Procedures, San Diego, Calif., vol. 3976, pp. 22-27, 2000.
[11] Holmes D, Davis B, Bruce C, Wilson T, and Robb R, “Trans-urethral ultrasound imaging of the prostate for applications in brachytherapy: analysis of phantom and in vivo data,” Proceedings SPIE Medical Imaging 2001: Visualization, Image-Guided Procedures, San Diego, Calif., vol. 4319, pp. 46-52, 2001.
[12] Davis B, Wilson T, Mynderse L, Kadri M, Dull D, Barnes S, Hangiandreaou N, Greenleaf J, Robb R, and Holmes D, “Initial clinical experience and design of a custom trans-urethral ultrasound probe for prostate imaging and guidance during minimally invasive prostate procedures,” Urology, vol. 68(Supplement 5A), pp. 302-303, 2006 (Abstract).
[13] Holmes D R, Davis B J, Hangiandreou N J, Kinnick R R, Wilson T M, Mynderse L A, and Robb R A, “Design and testing of a custom transurethral ultrasound device with rapid 3D reconstruction using a rotational scanner,” Brachytherapy, vol. 6, p. 96, 2007 (Abstract).

### SUMMARY

A three-dimensional transurethral ultrasound system according to an embodiment of the current invention includes a transurethral ultrasound probe, an ultrasound data processor configured to communicate with the transurethral ultrasound probe to receive ultrasound signals from the transurethral ultrasound probe and to output ultrasound imaging signals for three-dimensional ultrasound images of at least a portion of a patient's prostate, and a display system configured to communicate with the ultrasound data processor to receive the ultrasound imaging signals and to render three-dimensional ultrasound images of the at least the portion of the patient's prostate from the ultrasound imaging signals.

An ultrasound probe according to an embodiment of the current invention includes a catheter having a distal end and a proximal end, and a transducer array attached to the distal end of the catheter. The transducer array includes a plurality of transducer elements arranged in a substantially cylindrical array pattern concentrically with a longitudinal axis of the catheter.

### BRIEF DESCRIPTION OF THE DRAWINGS

Further objectives and advantages will become apparent from a consideration of the description, drawings, and examples.

FIG. 1 is a schematic illustration showing transducer placement during transrectal ultrasound (TRUS). Ultrasound energy must travel through the rectum wall and across the full volume of the prostate.

FIG. 2 is a schematic illustration showing transducer placement during transurethral ultrasound (TUUS) according to an embodiment of the current invention. The transducer can obtain imagery from directly within the prostate.

FIG. 3 shows several views of a cylindrical transducer array according to an embodiment of the current invention. This is effectively a two-dimensional transducer array used to produce a three-dimensional ultrasound image.

FIG. 4 is a schematic illustration of a real-time, three-dimensional transurethral ultrasound system according to an embodiment of the current invention.

FIG. 5 is a schematic of a real-time, three-dimensional transurethral ultrasound system according to an embodiment of the current invention.

FIG. 6 is a schematic illustration of a conformal transducer cross-section (top) and top view (bottom) according to an embodiment of the current invention.

FIG. 7 shows an example of a partially completed transducer arrays mounted in silicon wafer with polyimide joints connecting silicon islands.

FIG. 8 shows an example of a transducer with PZT attached to the silicon islands and flexible Au ground electrodes (inset) evaporated onto a parylene layer supporting the PZT and polyimide according to an embodiment of the current invention. Row of Al pads at bottom serve as signal pads.

FIG. 9 shows an example of a conformal array wrapped around a 9 F (3 mm diameter) commercial intracardiac ultrasound catheter according to an embodiment of the current invention.

FIG. 10 shows electrical impedance of the transducer, with a fundamental resonance at 12 MHz, according to an embodiment of the current invention.

FIG. 11 shows simulated reconstruction of nine adjacent point-targets (right) imaged outwardly by a 16 element cylindrical transducer array (left).

FIG. 12 shows a prototype with ribbon cable interconnect according to an embodiment of the current invention.

FIG. 13 shows an enclosed TUUS transducer array according to an embodiment of the current invention with a bladder catheter for comparison.

FIG. 14 shows an example of system electronics with an enclosure according to an embodiment of the current invention.

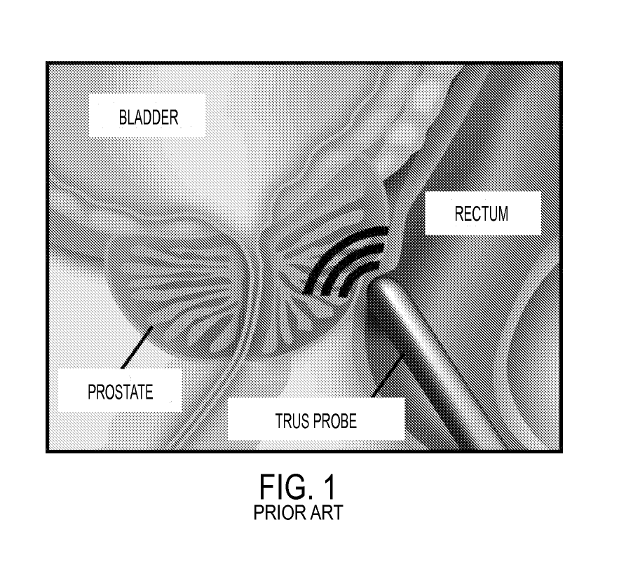

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

A transurethral catheter-based ultrasound system for imaging the prostate in three dimensions (3D) and for assisting with focal therapy and needle-based procedure through MR-CT-US fusion is provided according to some embodiments of the current invention. In some embodiments, this can be performed in real time. Multiple zones of the prostate can be imaged with ultrasound and registered to other modalities. In some embodiments, this can also be performed in real time. Focal therapy of prostate cancer requires accurate localization of brachytherapy needles or radiation beams. Examples of areas of use can include prostate brachytherapy, external beam radiation therapy, cryotherapy, and targeted biopsy, for example. Other examples can include, but are not limited to, Prostate volume estimation, surgical planning, and TURP.

Transducer technology currently exists with a radially-phased single-column intravascular ultrasound transducer from Volcano therapeutics, and with 2D cylindrical arrays mounted on an endorectal probe from Philips. See for example, the following:
US 2010/0137721 A1, US 2011/0137148 A1, US 2010/0241002 A1, US 2009/0048515 A1, U.S. Pat. No. 6,899,682;
Culjat, M. et al, “Transurethral Ultrasound Catheter-Based Transducer with Flexible Polyimide Joints”, IEEE Ultrason. Symp 2009; and
Holmes, D. et al, “3D visualization, analysis, and treatment of the prostate using trans-urethral ultrasound”. Computerized Medical Imaging and Graphics 27, 2003.

Embodiments of the current invention do not require mechanical manipulation to capture images, and instead acquire a 3D ultrasound image through electronic steering. For the prostate, existing devices which require rotation or mechanical translation introduce motion errors which must be corrected through software and require registration of N 2D ultrasound slices. A multi-row transducer array according to an embodiment of the current invention can allow real-time acquisition of 3D ultrasound natively. This real-time volume, in turn, can enable real-time image fusion with an MRI or CT volume acquired previously. The hardware transceiver connected to the catheter transducer array can allow for differing frequency modulation on independent channels, which can allow the operator to use frequency-based ultrasound imaging techniques in addition to B-mode, and doppler ultrasound.

The imaging device according to an embodiment of the current invention is a transducer array with row-column leads, such that an entire row, entire column, or patches of elements can be activated at once. The transducers have sufficient bandwidth to allow for frequency sweeping. In an embodiment of the current invention, the array is made from a polyimide backing layer with Lead Zirconate Titinate (PZT) diced on the array. However, the broad concepts of the current invention are not limited to only PZT. Some embodiments can use piezoceramics, piezocomposites, CMUTs, sol-gel, or any other type of piezo material that is suitable for the particular application. A flexible copper circuit that includes the top electrode is connected across each row. The bottom electrodes can be connected across each column through wire bonding or a second flexible circuit. The elements are then curved cylindrically around a rod backing layer.

A bank of multiplexers are placed adjacent to the array to allow for transducer elements to be sonicated by activating the appropriate rows and columns. Column multiplexers are electrically grounded to the bottom electrode so an activation causes the attached piezoelectric element to vibrate. The interconnect from the transducer/multiplexer interface to the hardware includes 8 twisted pairs, 2 per channel, and 5 addition control signals for the multiplexing circuit. These wires are electrically shielded and embedded in the catheter.

Transmitter electronics include a bank of 8 arbitrary waveform generators, which include a first-in-first-out buffer, a digital-to-analog converter, and amplifier. The receiver includes a low noise amplifier, low pass filter, and an octal analog-to-digital converter. A 3D image is acquired through sequentially firing through all transducer elements. The row-column multiplexing interface with the arbitrary-waveform hardware transceiver allows for M channels to be transmitting different ultrasound signals on N transducer elements, where N is the # of rows * # of columns. The entire system is controlled and attached to a PC via an FPGA and ethernet or USB connection.

After the 3D ultrasound image is acquired, software is used to outline the prostate in a set number of axial planes. This outline is used to create a surface mesh, which is then fused with a pre-loaded MRI, CT, PET, transrectal ultrasound, previously acquired TUUS image and/or other imaging modalities. During the procedure, the surface mesh is updated at fixed intervals or through manual input based on intensity variations between old and updated 3D volumes. The old transformation from ultrasound-MR or ultrasound-CT is re-applied to the new volumetric image. This transformation is then updated in real-time based on the surface map. During needle-based procedures, the real-time 3D ultrasound image can be used to determine the needle deflection during insertion.

Some embodiments of the current invention can be used in conjunction with prostate cancer therapy (brachytherapy, external beam radiation) or during diagnostic biopsy (for lesion targeting with MRI). A patient who undergoes focal therapy for prostate cancer as an alternative to surgery would receive an MRI to identify a suspicious area or an area with an existing diagnosis of cancer. A CT would be obtained for radiation beam alignment. The catheter transducer is inserted into the patient through the urethra, and advanced until the prostate is in view on the monitor. The catheter location is fixed during the procedure. The boundaries of the prostate are then identified and traced on the attached computer and monitor. An MRI or CT acquired previously is then loaded into the system via CD, USB, or a PACS link, and registered with the ultrasound volume. Registration can be performed in a 2D manner by selecting corresponding points on representative scanning planes (axial, sagittal, coronal), or in 3D via existing software methods.

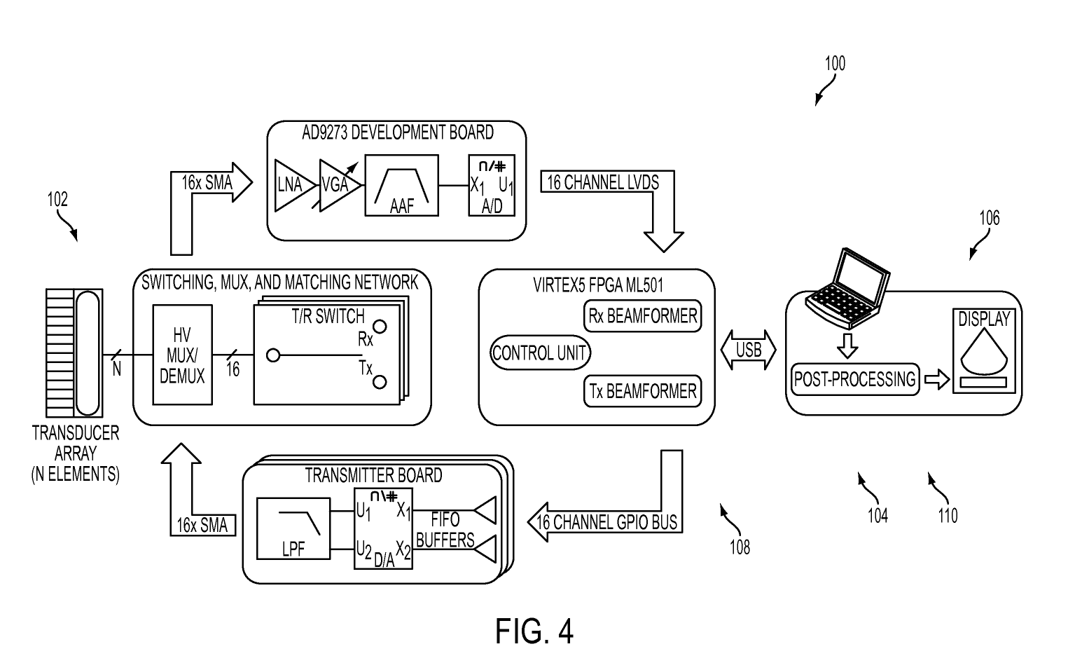

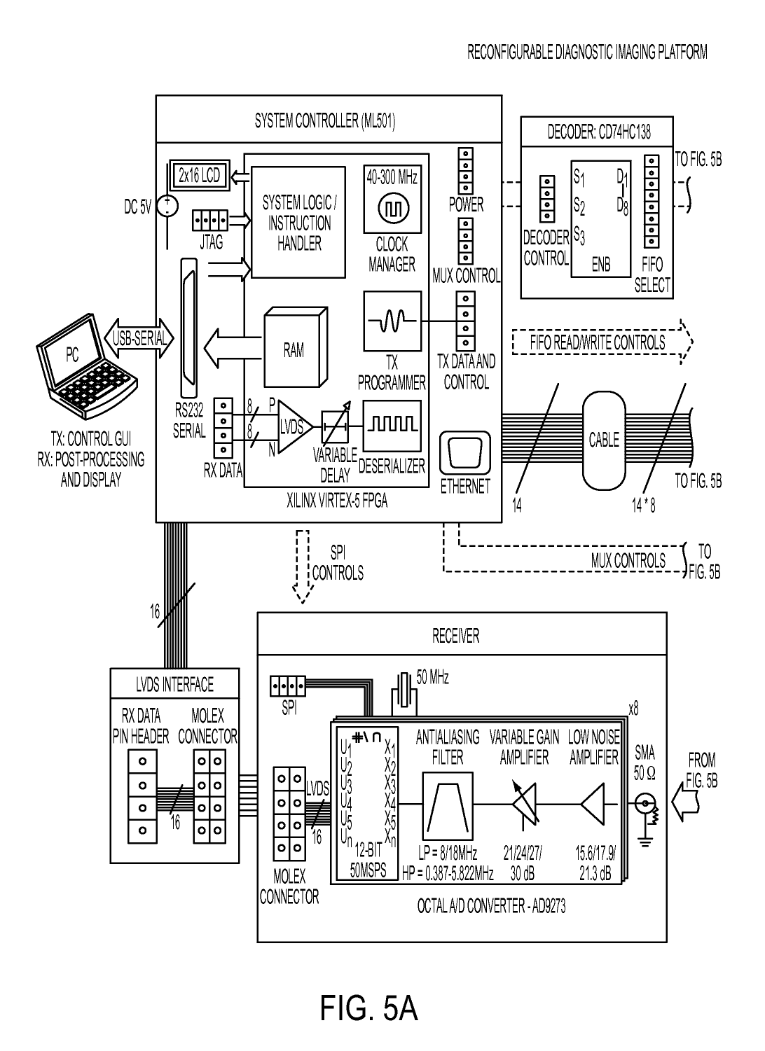

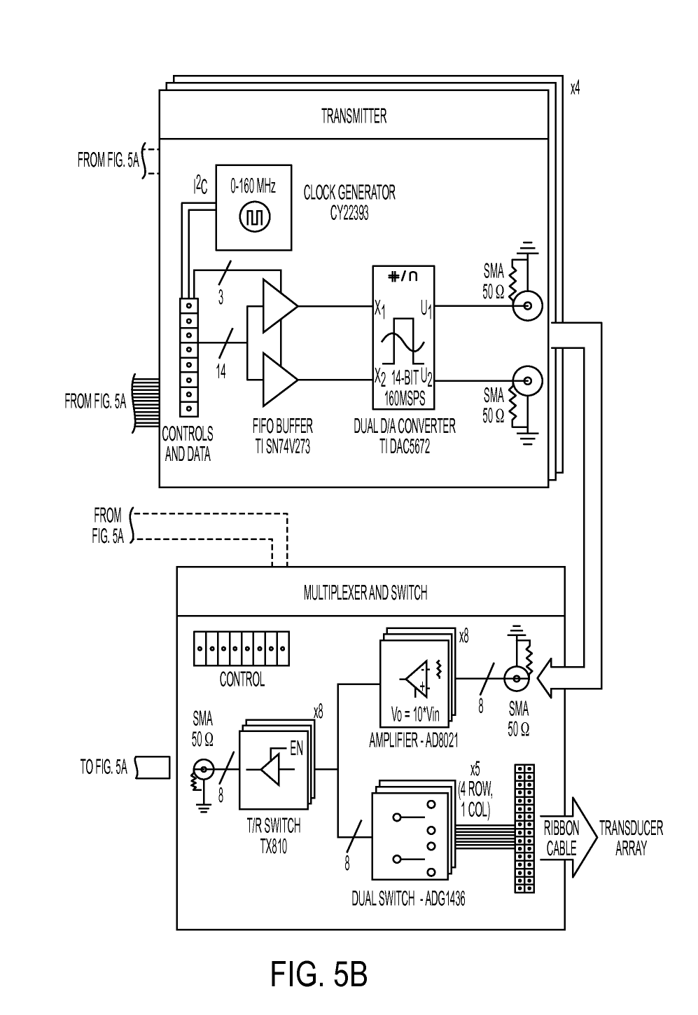

FIG. 4 is a schematic illustration of a three-dimensional transurethral ultrasound system 100 according to an embodiment of the current invention. The three-dimensional transurethral ultrasound system 100 includes a transurethral ultrasound probe 102, an ultrasound data processor 104 configured to communicate with the transurethral ultrasound probe 102 to receive ultrasound signals from the transurethral ultrasound probe 102 and to output ultrasound imaging signals for real-time, three-dimensional ultrasound images of at least a portion of a patient's prostate. The three-dimensional transurethral ultrasound system 100 also includes a display system 106 configured to communicate with the ultrasound data processor 104 to receive the ultrasound imaging signals and to render three-dimensional ultrasound images of at least a portion of the patient's prostate in real time from the ultrasound imaging signals.

In some embodiments, the three-dimensional transurethral ultrasound system 100 can be a real-time, three-dimensional transurethral ultrasound system. The term “real time” is intended to mean that the images are displayed sufficiently rapidly to be used during a medical procedure concerning the patient's prostate. This can include, but is not limited to, real-time use during diagnosis, biopsy and/or treatment. This can include, but is not limited to, real-time use to assist with tracking and/or guidance of surgical tools, for example.

The three-dimensional transurethral ultrasound system 100 can also include transceiver electronics 108, which will be described in more detail below for some particular embodiments.

The ultrasound data processor 104 can be implemented on a computer and/or networked computers, for example. It can be implemented through software to program the computer and/or dedicated hardware such as, but not limited to, FPGA's and/or ASIC's. In some embodiments, the three-dimensional transurethral ultrasound system 100 can also include an image registration unit 110 configured to receive preoperative image data and register the preoperative image data with the three-dimensional ultrasound images. In some embodiments, data other than preoperative data can be registered with the three-dimensional ultrasound images, such as, but not limited to model data and/or real time data from other imaging modalities. In some embodiments, the image registration unit 110 can perform the image registration in real time. The image registration unit 110, similar to the ultrasound data processor 104, can be implemented through software to program the computer and/or dedicated hardware such as, but not limited to, FPGA's and/or ASIC's. In some embodiments, the image registration unit 110 can be implemented on the same computer, or networked computers, as the ultrasound data processor 104.

In some embodiments, the preoperative image data can be at least one of magnetic resonance imaging (MRI) data, x-ray computed tomography (CT) data, positron emission tomography (PET) data, or single-photon emission computed tomography (SPECT) data.

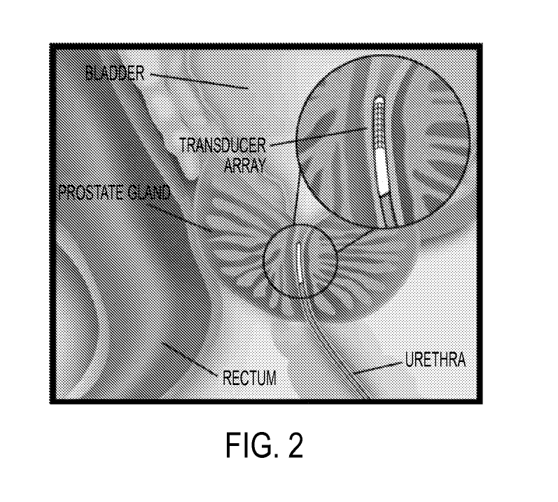

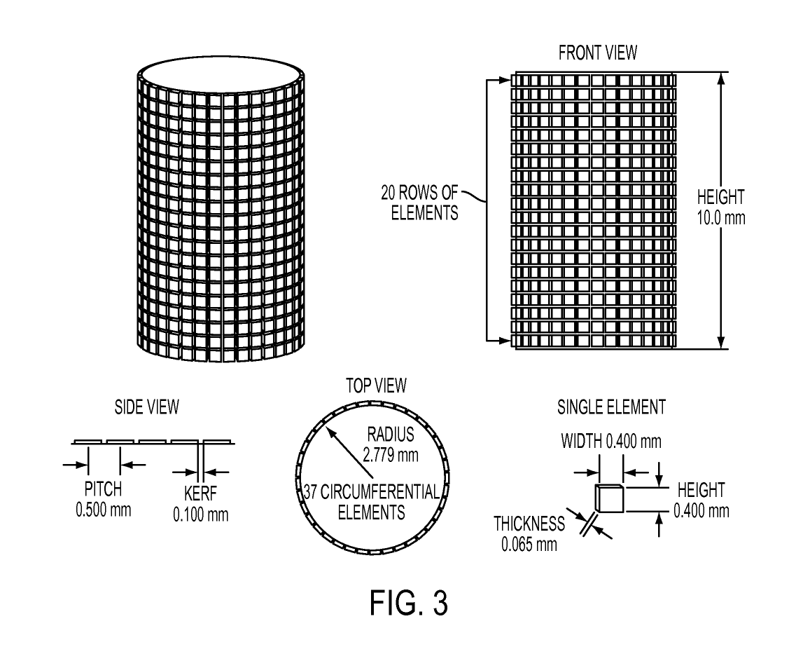

In some embodiments, the transurethral ultrasound probe 102 includes a catheter that has a distal end and a proximal end, and a transducer array attached to the catheter proximate said distal end of the catheter (FIG. 2). The catheter can be rigid, semi-rigid, or flexible. The transducer array includes a plurality of transducer elements arranged in a substantially cylindrical array pattern concentrically with a longitudinal axis of the catheter. FIG. 3 shows an example of a transducer array according to an embodiment of the current invention. In this example, there are 20 rows of 37 elements in each row for an array that is 1 cm long along the axial direction (i.e., the height of the cylindrical array). The general concepts of the current invention are not limited to this example. In some embodiments, there may be as few as two rows. In other embodiments, it can be useful to provide an array up to 3 cm long. In some embodiments, the transducer array can include at least 32 transducer elements arranged in at least two rows circumferentially around said catheter.

The size of each element of the array can be selected according to the desired transmission/reception frequency, for example. In some embodiments, each of the plurality of transducer elements has a surface area of at least 4×10⁻⁸ m² and less than 36×10⁻⁸ m². In some embodiments, each of the plurality of transducer elements has a surface area of about 16×10⁻⁸ m².

In some embodiments, the catheter of the transurethral ultrasound probe 102 can be at least 1 mm wide and less than about 8 mm wide. In some embodiments, the catheter of the transurethral ultrasound probe 102 can be about 6 mm wide.

In some embodiments, an ultrasound probe can have the same or similar structure as the transurethral ultrasound probe 102. However, it can be used for other procedures with other organs, for example being inserted intravenously and/or some other minimally invasive surgical procedure. The size of the catheter, the transducer array and/or transducer elements can be selected for optimization for the particular application.

### EXAMPLES

The following examples help explain some concepts of the current invention. However, the general concepts of the current invention are not limited to the particular examples.

### Example 1

This example according to an embodiment of the current invention describes an approach that has the potential to eliminate rotation and pull-back in TUUS and provide improved 3D imagery by using a 2D cylindrical transducer array. A TUUS transducer prototype was developed that features a cylindrical array of piezoelectric elements that can be embedded within or wrapped around a catheter body. Fabrication of the transducers was made possible with a highly durable MEMS-based microfabrication process (Bennett D B, Culjat M O, Cox B P, Dann A E, Williams K, Lee H, Brown E R, Grundfest W S, and Singh R S, “A Conformal ultrasound transducer array featuring microfabricated polyimide joints” Proceedings of SPIE Health Monitoring of Structural and Biological Systems III, 8-12 Mar. 2009, San Diego, Calif., vol. 7295, pp. 72951W, 2009) that allows the array to be fabricated in a planar configuration to achieve dense element spacing, and to subsequently be rolled to the desired curvature. This technique greatly simplifies the fabrication of cylindrical transducer architectures, while also allowing compact size. The technique also permits scaling to various array sizes and element configurations, such that it can accommodate a variety of catheter sizes and designs. The flexibility of the transducer not only allows it to be rolled to a cylindrical configuration, but also allows the transducer to bend, following and conforming to the curved contours of the urethra.

### Transducer Design

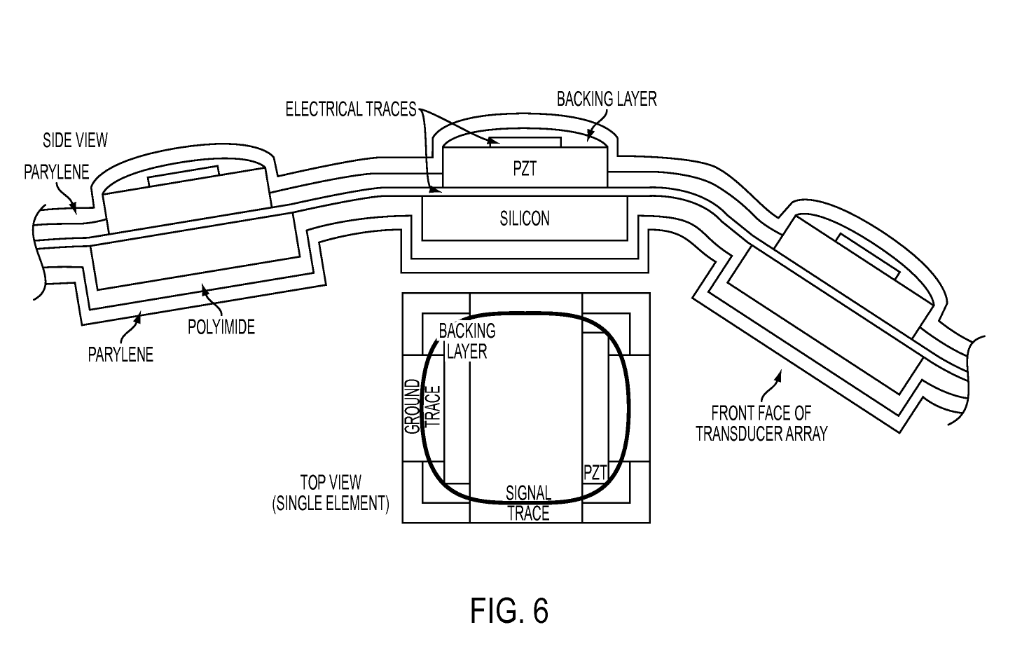

The transducer design features bulk lead zirconate titanate (PZT) elements mounted on an array of silicon islands formed by partitioning a silicon wafer via deep reactive ion etching (DRIE) (FIG. 6). Polyimide was patterned to form bendable joints, which allow substrate flexibility and encapsulate metal film interconnects. The silicon islands fulfill the dual roles of mechanical substrate and acoustic matching layer to soft tissue in conjunction with a deposited parylene film. Polyimide was selected because it is mechanically strong, readily patternable by photo-lithographic techniques, and resistant to chemicals used in microfabrication processes. A two layer electrode network was incorporated for row-column addressing of the piezoelectric elements, minimizing the number of traces. The process was adapted from one originally developed for conformal inward-looking ultrasound arrays (Culjat M O, Bennett D B, Lee M, Brown E R, Lee H, Grundfest W S, and Singh R S, “Polyimide-based conformal ultrasound arrays for image guidance” IEEE Sensors Journal, 9(10), 1244-1245, 2009; Singh R S, Culjat M O, Lee M, Bennett D B, Natarajan S, Cox B P, Brown E R, Grundfest W S and Lee H, “Conformal ultrasound imaging system,” Acoustical Imaging 30, 1-4 Mar. 2009, Monterrey Calif.).

The prototype transducer featured bulk PZT elements that were attached to the substrate using alignment trenches. The microfabrication process was designed to support other alternative placement techniques such as dice and fill and flip-chip bonding, which may be used to produce higher density arrays. Many thin-film deposition techniques have recently been proposed for 2D array transducers, including sol-gel thin-film piezoelectric micromachined ultrasound transducers (PMUTs) and capacitive micromachined ultrasound transducers (CMUTs). However, bulk piezoelectric materials were selected for this embodiment due to their high electroacoustic efficiency and low drive voltages, both necessary for transurethral imaging of the prostate. Enlarged prostates may be more than 7 cm in diameter, therefore requiring up to 4 cm penetration to image the full prostate volume (Eri, L M, Thomassen H, Brennhovd B, and Hahheim L L, “Accuracy and repeatability of prostate volume measurements by transrectal ultrasound,” Prostate Cancer and Prostate Diseases, vol. 5, pp. 273-278, 2002).

### Transducer Fabrication

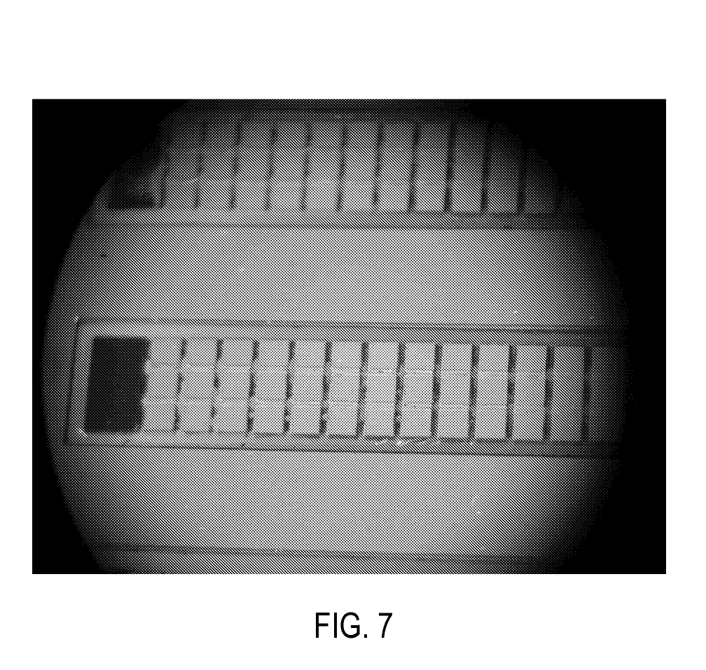

A silicon wafer was sectioned via deep reactive ion etching (DRIE) to produce an array of silicon mesas. A polyimide layer was spin coated and cured onto the bottom side of the wafer. The silicon on the top-side of the wafer was etched to release a flexible substrate of silicon islands with polyimide joints. An aluminum electrode film was sputtered and patterned onto the flexible substrate, and an additional polyimide layer was cured and patterned atop the wafer to encapsulate and protect the electrodes, reinforce the flexible joints, and serve as alignment trenches for the placement of piezoelectric elements on the silicon islands (FIG. 7).

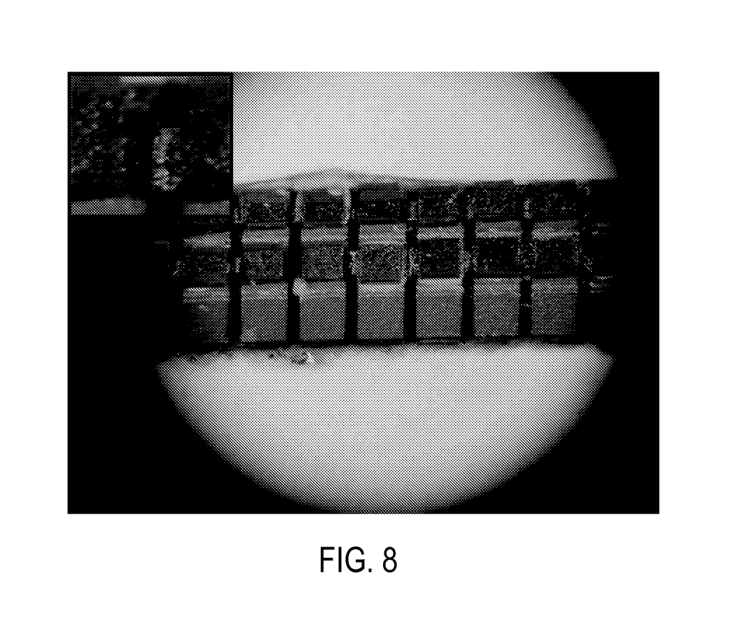

Ceramic PZT elements were diced and attached to the silicon islands using thin layers of silver conductive ink, and a conformal parylene layer was added and patterned to both mechanically secure and electrically isolate the bottom electrode and traces of the PZT. Via holes were etched into the top PZT electrodes using oxygen plasma. A second set of electrical leads composed of chromium and gold were patterned by electron beam evaporation through a shadow mask of Kapton film, connecting to the top electrode through the via holes in the parylene (FIG. 8). A tungsten-loaded epoxy acoustic backing layer and an additional parylene film were then added.

### Transducer Characterization

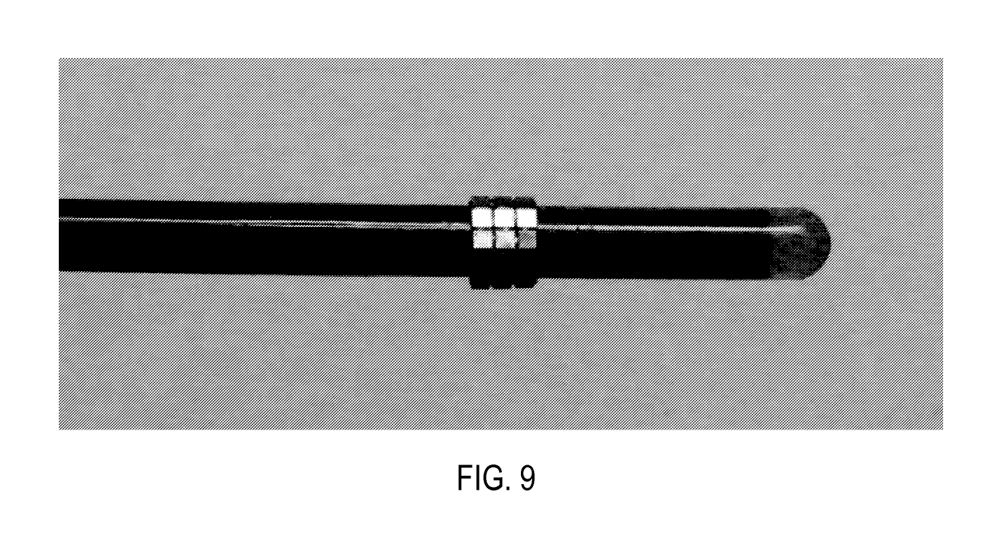

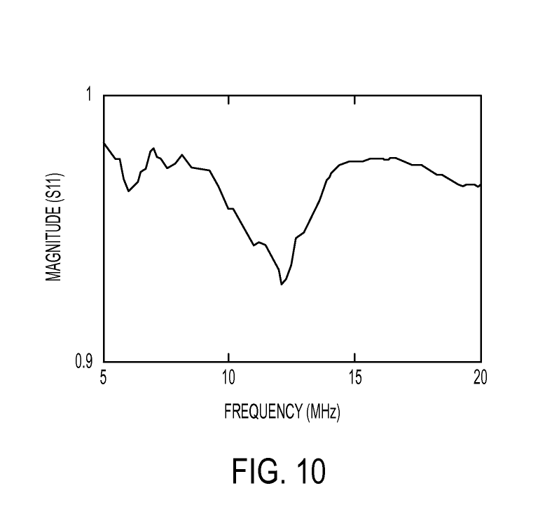

A prototype 2×16 element TUUS transducer is shown wrapped around a 9 F (3 mm diameter) intracardiac ultrasound catheter in FIG. 9. The transducer has a 400 μm element width, 500 μm pitch, and a pulse width of 300 ns in water. The prototype transducer was electrically characterized using a vector network analyzer (VNA), revealing a 12 MHz resonant frequency and a 3 MHz 6 dB bandwidth (FIG. 10). The transducer could be rolled to smaller than a 2 mm radius of curvature (dependent only on element size and pitch). The polyimide/parylene joints were demonstrated to withstand more than 10,000 bending cycles without visible damage or measurable deterioration of the interconnects.

This example is intended for fitting onto an 18 F (6 mm diameter) catheter, a size commonly used for transurethral procedures. This initial two row array was produced to determine the viability of the 2D array fabrication approach as well as the row-column addressing technique. The process can be scaled to larger 2D arrays of smaller element dimensions, which can enable volumetric imaging and fitting onto smaller 9 F catheters.

### Transducer Simulation

An example image reconstruction was generated to demonstrate the ability to capture imagery using a non-phased cylindrical transducer array and to characterize the potential resolving capability in the prostate environment. The image reconstruction is based on the backward propagation method, a numerical migration technique capable of reconstructing the source distribution from scattered wave-field data and producing high-resolution images (Brown A and Lee H, “Backward Propagation Image Reconstruction Techniques for Bistatic Synthetic-Aperture Radar Imaging Systems with Circular-Aperture Configurations,” Proceedings of the 35th Asilomar Conference on Signals, Systems and Computers, pp. 110-115, 2001). This algorithm can be implemented in a parallel-processing scheme for real-time imaging and has been shown to be capable of handling large matrices of data that would be collected from the 2D cylindrical arrays.

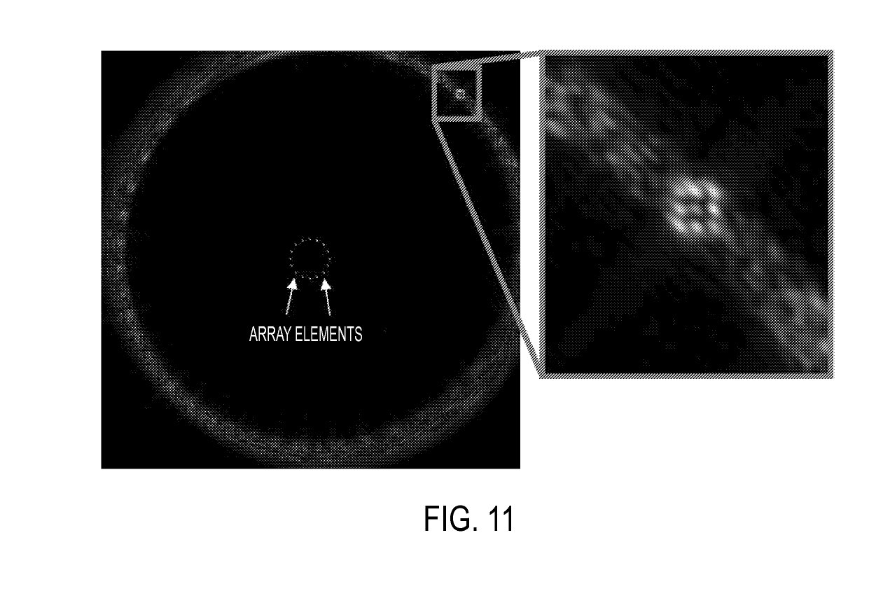

A Matlab model was developed with a circular array of 16 elements operating at 12 MHz with a 3 MHz bandwidth and diameter of 6 mm (18 F). A distribution of nine point targets spaced 0.4 mm apart were placed in the model 3 cm from the transducer. The reconstructed image (FIG. 11, left) was generated by cycling individual elements through to transmit an idealized pulse, during which all other elements were set to receive mode. The resulting A-scans were mapped onto a 2D range bin matrix, producing the image. The image represents a 6 cm×6 cm region of interest, imaged with a 6 mm diameter (18 F) transducer array. The observed background interference patterns encircling the image are due to the finite bandwidth of the illumination signal and array configuration. The distribution of targets (FIG. 10, right) was resolved, even with the relatively sparse 16 element cylindrical array and 3 cm range. Further simulation will be performed to determine the optimal number of elements, transducer dimensions, and pitch for various catheter diameters.

A transducer fabrication technique has been developed for 2D cylindrical catheter-mounted arrays for TUUS. When optimized and integrated with an imaging system, the transducer is expected to provide high quality imagery of the transition zone and anterior prostate, allow for volumetric assessment of the prostate, and deliver more accurate therapeutic interventions, including radioactive seed placement and prostate biopsies. The technology allows for scaling to various catheter sizes, and therefore may also find application to a variety of urologic, cardiovascular, and nondestructive testing applications.

### Example 2

### Prostate Imaging

Multi-modal imaging techniques, which involve the fusing of Ultrasound images and CT or MRI images on a consistent coordinate system, are promising as real-time tools for the urologist. Through the registration of the real-time imaging capabilities of ultrasound and the increased information density offered by MRI, prostate biopsies can potentially take advantage of both imagining modalities. However, these fusion techniques still rely on TRUS imaging, which is susceptible to artifacts developed in acoustic waves traveling through the rectal wall, and can suffer from poor resolution of the anterior prostate. Additionally, current 3D TRUS-based imaging techniques, required for fusion, are susceptible to large motion artifacts due to external placement of the probe in relation to the prostate.

In an effort to address several of these shortcomings, transurethral ultrasonography (TUUS) has been proposed as an alternative to TRUS in real-time prostate imaging [8]. Due to the urethra's position within the center of the prostate, TUUS can capture a uniform image of the entire gland from the inside. Additionally, the decreased penetration depth required by TUUS allows higher frequencies to be used, resulting in potentially improved resolution. Moreover, patient motion is less influential on image quality, due to the central location of TUUS transducers within the prostate.

Several past attempts have been made to realize TUUS imaging, but have all been limited in their ability to produce high-quality 3D images of the prostate. Some embodiments of the current invention provide a novel transurethral prostate-imaging system capable of 3D imaging with a cylindrical multi-dimensional transducer array, as well as configurable system electronics capable of multi-channel data processing. Real-time 3D visualization of the prostate should facilitate the fusion of MR and US images, enabling lesion imaging with MR to be overlaid with real-time TUUS-guided biopsy imaging. A number of engineering challenges have been addressed to bring this concept to realization: a catheter-based transducer capable of volumetric imaging of the prostate was fabricated and evaluated; custom hardware was designed to provide flexibility in applying pulse-echo based and frequency-based imaging methods; and optimized image processing techniques were developed and implemented for high-resolution image reconstruction and MR-US fusion in 3D.

### Methods & Materials

The potential capabilities of TUUS imaging of the prostate were demonstrated in a series of papers by D. R. Holmes et al. between 2000 and 2004. These studies investigated the feasibility of a TUUS system using commercial intravascular catheter-based transducers, featuring single rotational elements and linear transducer arrays [8,9]. This system was capable of 3D imaging, but required manual rotation of the linear transducer array [8,9], and required constant-velocity translation of the single element transducer [8]. In addition to the discomfort that manual catheter manipulation introduces, non-uniform motion contributes distortions during image-capture. Despite these limitations, in-vivo studies in canines found that TUUS was capable of accurately localizing radioactive seeds and prostate boundaries, while making improvements in resolution to both CT and TRUS [10]. The system in this example can improve upon this concept in two fundamental ways: (1) integration of a 2D transducer array to enable real-time 3D imaging of the prostate, and (2) use of frequency-based signal processing methods to improve the visualization of features of clinical interest. Additionally, this system was designed as a flexible platform facilitating future imaging studies, such as those implementing multi-modal fusion. The system's ability to capture real-time 3D ultrasound information enables co-registration of volumetric data obtained with other medical imaging modalities through the implementation of standard registration techniques. Furthermore, the system was designed with the capability of scaling with the number of transducer elements.

### Transducer Design

An initial prototype of the cylindrical 2D transducer array was constructed similarly to a previously described micro fabrication process [12]. A flexible polyimide substrate was used to allow for bending and wrapping in a cylindrical shape. Copper leads were patterned on the PI-substrate. Lead zirconate titanate (PZT) plates with gold electrodes were soldered to the copper layer, which were then diced in single elements forming the array. The entire array was then wire-bonded, wrapped, and filled with a backing layer (PDMS). The array elements were patterned in a row-column arrangement, to simplify interconnections and to allow for scalability with multiplexers. The initial transducer array contained 2 rows of 16 elements with a diameter of 6 mm, with a frequency of operation at 18 MHz. This high initial frequency was chosen to test the range of the system electronics. An optimal design for the prostate requires a 9-12 MHz array. A ribbon cable containing twisted pairs of conductors was soldered onto 18 leads (one for each row and column) patterned on the transducer array. The cable terminated with a standard connector for compatibility with the system electronics.

### Configurable Transceiver Platform

A custom transceiver platform was designed to enable functionality independent of the signal processing techniques used, the transducer type, or the frequency used. A Xilinx ML501 FPGA development board functions as the system's central controller, responsible for synchronization across modules, digital processing, and communication between the PC over USB. The use of an FPGA allows direct control over the implementation of the various system functions at a hardware logic level. Additionally, an FPGA provides flexibility in accommodating changes that may be made to hardware interfaces or imaging mechanisms for future studies. Eight parallel arbitrary waveform generators serve as the transmitter component, allowing for per-channel variation in frequency, phase, signal pattern, and modulation. These transmitters are capable of imaging at frequencies up to 20 MHz, with a signal resolution of 160 MSPS. The use of a decoder interface between the transmitter and the FPGA enables transmitter scalability with minimal modification to other hardware components. A multi-channel receiver (AD9273) is responsible for amplifying, and filtering, and digitizing the received signal for further processing and visualization. The system supports eight channels of parallel data acquisition with the capability of connecting additional receiver modules. Received data is digitized at a rate of 50 MSPS with a resolution of up to 12 data bits. The transducer interface is capable of mono- or multi-static transmission, and can multiplex up to 64 channels. The flexible hardware configuration allows both time- and frequency-based transmission and post-processing techniques to be easily implemented, requiring only FPGA reprogramming. This capability facilitates the analysis of system performance under varying ultrasound signaling techniques, including b-mode, frequency-modulated continuous wave (FMCW) variants, and synthetic aperture imaging.

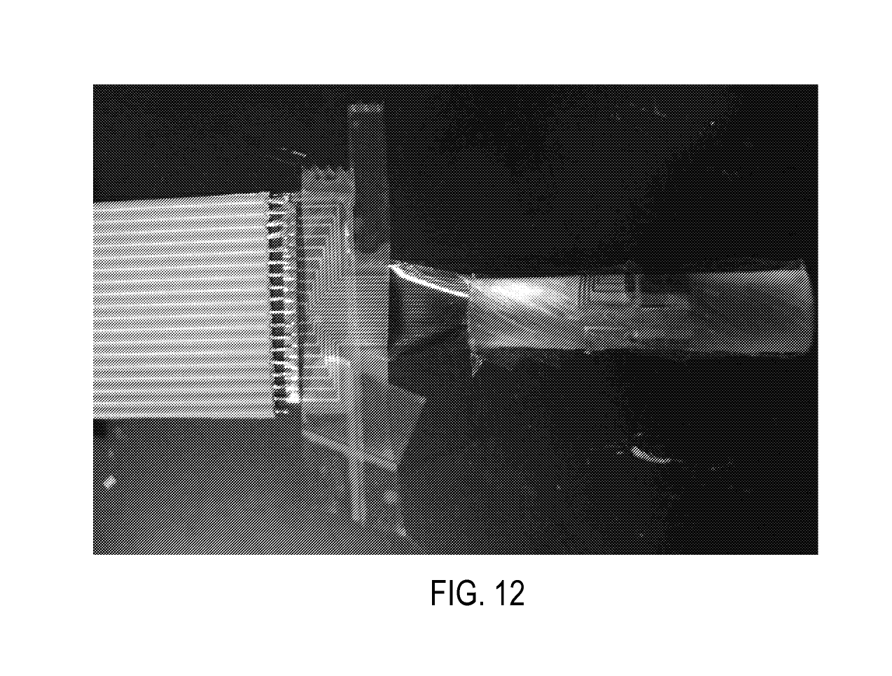

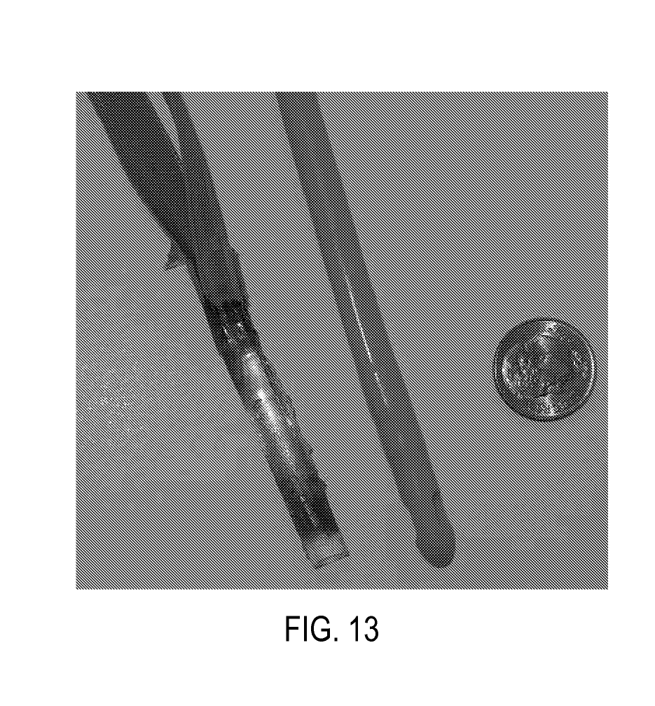

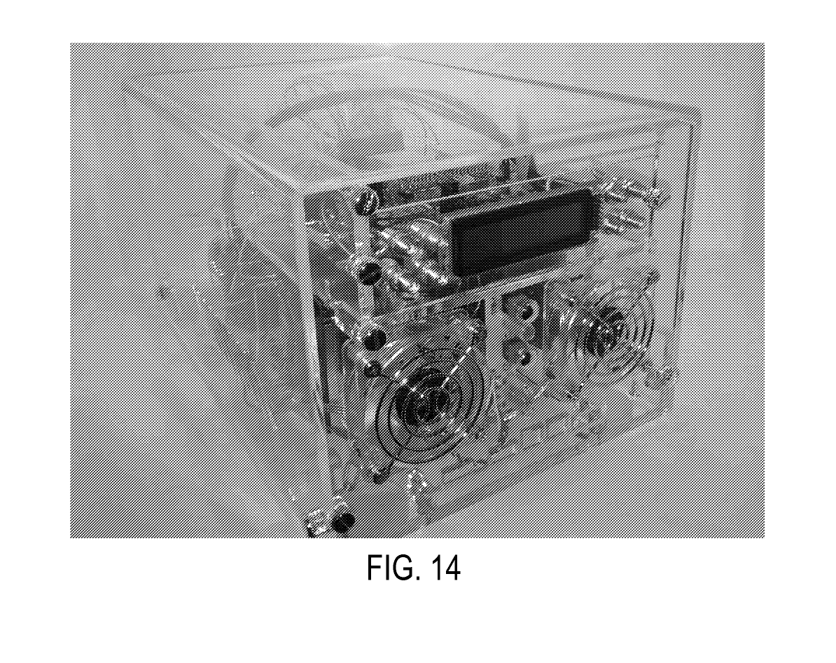

A prototype of the catheter-based transducer array has been completed, using the MEMS-based process (FIGS. 12), consisting of 32 elements with a center frequency of 18 MHz. Pulse-echo data was successfully obtained using the prototype transducer (FIG. 13) and transceiver, demonstrating this approach. The hardware has been realized (FIG. 14) and tested for the ability to output both pulse-echo and frequency-based transmission patterns. Transducers with larger array dimensions can be used.

TUUS can potentially improve upon existing prostate imaging techniques, such as TRUS, for applications in diagnosis and therapy. By taking advantage of the urethra's central locality within the prostate, TUUS can improve 3D ultrasonic imaging of the prostate for multi-modal applications. A robust system that allows for quantifiable comparisons between different signaling and post-processing techniques has been developed.

### REFERENCES FOR EXAMPLE 2

[1] Jemal A, Bray F, Center M M, Ferlay J, Ward E, Forman D, 2011, “Global cancer statistics,” CA Cancer J Clin, vol. 60, pp. 277-300.
[2] Silletti J P, Gordon G J, Bueno R, Jaklitsch M, Loughlin K R, 2007, “Prostate biopsy: Past, present, and future,” J Urology, vol. 69(3), pp. 413-6.
[3] Turkbey B, Xu S, Kruecker J, Locklin J, Pang Y, Bernardo M, Merino M J, Wood B J, Choyke P L, Pinto P A. Documenting the location of prostate biopsies with image fusion. BJU Intl 2010; 107:53-57.
[4] Kitajima K, Kaji Y, Fukabori Y, Yoshida K, Suganuma N, Sugimura K. Prostate cancer detection with 3 T MRI: Comparison of diffusion-weighted imaging and dynamic contrast-enhanced MRI in combination with T2-weighted imaging. J Magn Reson 2010; 31:625-631.
[5] Coakley F V, Qayyum A, Kurhanewic J, 2003, “Magnetic Resonance Imaging and Spectroscopic Imaging of Prostate Cancer,” Journal of Urology, vol. 170, pp. S69-S76.
[6] Dubois D F, Prestidge B R, Hotchkins L A, Prete J J, Bice W S, 1998, “Intraobserver and interobserver variability of MR imaging- and CT-derived prostate volumes after trasnperineal interstitial permanent prostate brachytherapy,” Radiology, vol. 207(3), pp. 785-789.
[7] Singh A K, Kruecker J, Xu S, Glossop N, Guion P, Ullman K, Choyke P L, Wood B J. Initial clinical experience with real-time transrectal ultrasonography-magnetic resonance imaging fusion-guided prostate biopsy. BJU international 2008; 101:841-845.
[8] Holmes D R, Robb R, 2000, “Trans-urethral ultrasound (TUUS) imaging for visualization and analysis of the prostate and associated tissues,” Proceedings SPIE Medical Imaging 2000: Visualization, Image-Guided Procedures, San Diego, Calif., vol. 3976, pp. 22-27.
[9] Holmes D, Davis B, Bruce C, Wilson T, Robb R, 2001, “Trans-urethral ultrasound imaging of the prostate for applications in brachytherapy: analysis of phantom and in vivo data,” Proceedings SPIE Medical Imaging 2001: Visualization, Image-Guided Procedures, San Diego, Calif., vol. 4319, pp. 46-52.
[10] Holmes D R, Davis B J, Bruce C J, and Robb R A, 2002, “3D visualization, analysis, and treatment of the prostate using trans-urethral ultrasound,” Computerized Medical Imaging and Graphics, vol. 27(5), p. 339
[11] Culjat M O, Dann A E, Lee M, Bennett D B, Schulam P G, Lee H, Grundfest W, Singh R S. Transurethral ultrasound catheter-based transducer with flexible polyimide joints. 2009, 2209-2212.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A three-dimensional transurethral ultrasound system, comprising:
a transurethral ultrasound probe;
an ultrasound data processor configured to communicate with said transurethral ultrasound probe to receive ultrasound signals from said transurethral ultrasound probe and to output ultrasound imaging signals for three-dimensional ultrasound images of at least a portion of a patient's prostate; and
a display system configured to communicate with said ultrasound data processor to receive said ultrasound imaging signals and to render three-dimensional ultrasound images of said at least said portion of said patient's prostate from said ultrasound imaging signals.

2. A three-dimensional transurethral ultrasound system according to claim 1, further comprising an image registration unit configured to receive preoperative image data and register said preoperative image data with said three-dimensional ultrasound images.

3. A three-dimensional transurethral ultrasound system according to claim 1, wherein said preoperative image data is at least one of magnetic resonance imaging data, x-ray computed tomography data, positron emission tomography data, single-photon emission computed tomography data, or ultrasound data.

4. A three-dimensional transurethral ultrasound system according to claim 1, wherein said transurethral ultrasound probe comprises:
a catheter having a distal end and a proximal end; and
a transducer array attached to said distal end of said catheter, and
wherein said transducer array comprises a plurality of transducer elements arranged in a substantially cylindrical array pattern concentrically with a longitudinal axis of said catheter.

5. A three-dimensional transurethral ultrasound system according to claim 4, wherein said catheter is at least 1 mm wide and less than about 8 mm wide.

6. A three-dimensional transurethral ultrasound system according to claim 4, wherein said catheter is about 6 mm wide.

7. A three-dimensional transurethral ultrasound system according to claim 4, wherein said plurality of transducer elements of said transducer array is at least 32 transducer elements arranged in at least two rows circumferentially around said catheter.

8. A three-dimensional transurethral ultrasound system according to claim 6, wherein said plurality of transducer elements of said transducer array is at least 32 transducer elements arranged in at least two rows circumferentially around said catheter.

9. A three-dimensional transurethral ultrasound system according to claim 8, wherein said transducer array is about 3 cm long along a direction of said longitudinal axis.

10. A three-dimensional transurethral ultrasound system according to claim 4, wherein each of said plurality of transducer elements has a surface area of at least 4×10⁻⁸ m² and less than 36×10⁻⁸ m².

11. A three-dimensional transurethral ultrasound system according to claim 4, wherein each of said plurality of transducer elements has a surface area of about 16×10⁻⁸ m².

12. A three-dimensional transurethral ultrasound system according to claim 9, wherein each of said plurality of transducer elements has a surface area of about 16×10⁻⁸ m².

13. A three-dimensional transurethral ultrasound system according to claim 1, wherein said ultrasound data processor is further configured to render said three-dimensional ultrasound images in real time.

14. A three-dimensional transurethral ultrasound system according to claim 2, wherein said image registration unit is further configured to receive preoperative image data and register said preoperative image data with said three-dimensional ultrasound images in real time.

15. An ultrasound probe, comprising:
a catheter having a distal end and a proximal end; and
a transducer array attached to said distal end of said catheter,
wherein said transducer array comprises a plurality of transducer elements arranged in a substantially cylindrical array pattern concentrically with a longitudinal axis of said catheter.

16. An ultrasound probe according to claim 15, wherein said catheter is at least 1 mm wide and less than about 8 mm wide.

17. An ultrasound probe according to claim 15, wherein said catheter is about 6 mm wide.

18. An ultrasound probe according to claim 15, wherein said plurality of transducer elements of said transducer array is at least 32 transducer elements arranged in at least two rows circumferentially around said catheter.

19. An ultrasound probe according to claim 17, wherein said plurality of transducer elements of said transducer array is at least 32 transducer elements arranged in at least two rows circumferentially around said catheter.

20. An ultrasound probe according to claim 19, wherein said transducer array is about 3 cm long along a direction of said longitudinal axis.

21. An ultrasound probe according to claim 15, wherein each of said plurality of transducer elements has a surface area of at least 4×10⁻⁸ m² and less than 36×10⁻⁸ m².

22. An ultrasound probe according to claim 15, wherein each of said plurality of transducer elements has a surface area of about 16×10⁻⁸ m².

23. An ultrasound probe according to claim 20, wherein each of said plurality of transducer elements has a surface area of about 16×10⁻⁸ m².
